(19) United States
(12) Patent Application Publication
(10) Pub. No.: US 2019/0159702 A1
(43) Pub. Date: May 30, 2019

(54) Methods for Correcting Otoacoustic Emission Measurements

(71) Applicant: Massachusetts Eye and Ear Infirmary, Boston, MA (US)

(72) Inventors: Karolina Charaziak, Somerville, MA (US); Christopher Shera, Belmont, MA (US)

(21) Appl. No.: 16/096,635

(22) PCT Filed: Apr. 28, 2017
(86) PCT No.: PCT/US17/30020

§ 371 Date: Oct. 25, 2018

Related U.S. Application Data
(60) Provisional application No. 62/328,881, filed on Apr. 28, 2016.

(51) Int. Cl. A61B 5/12; A61B 5/00

(52) U.S. Cl. CPC A61B 5/12; G01H 15/00; A61B 5/6817

(57) ABSTRACT

The methods disclosed herein enable calculating otoacoustic emission (OAE) pressure independent of the acoustic load imposed by the ear canal and the OAE probe measurement system, e.g., for hearing tests. The OAE pressure is calculated in a form of either the first outgoing wave at the eardrum, referred as emitted pressure level (P_{EPL}), or as a Thvenin-equivalent OAE source pressure level (P_{TPL}) at the eardrum, as derived from a simple tube model of an ear canal. In both methods the OAE sound pressure level (P_{SPL}), ear canal reflectance (R_{EC}), OAE probe source reflectance (R_{S}), and one-way ear canal delay (τ) are measured at the entrance of the ear canal with the OAE probe. In contrast to P_{SPL}, both methods result in an emission pressure that is not confounded by the effects of the residual ear canal space or the impedance of the OAE measurement system.

### CROSS-REFERENCE TO RELATED APPLICATIONS

This application claims priority to U.S. Patent Application 62/328,881 filed on Apr. 28, 2016, the entire contents of which are incorporated herein by reference.

### STATEMENT AS TO FEDERALLY SPONSORED RESEARCH

This invention was made with Government support under R01 DC003687 awarded by the National Institutes of Health. The Government has certain rights in the invention.

### TECHNICAL FIELD

This disclosure relates to ear-canal measurements of otoacoustic emissions (OAEs) (sounds generated by the inner ear), and more particularly to methods of correcting otoacoustic emissions for ear-canal acoustics.

### BACKGROUND

OAEs have been used primarily as a way to monitor and/or assess the health of the inner ear noninvasively in both clinical and laboratory settings and can provide an advance warning of impending permanent hearing loss, e.g., in persons exposed to excessive sound levels. For example, the level of the OAE from the ear can start to drop even before a noticeable hearing loss appears (see, Marshall et al., “Detecting incipient inner-ear damage from impulse noise with otoacoustic emissions,” J. Acoust. Soc. Amer., 125(2):995-1013 (2009)). Permanent hearing loss can be predicted by low-level or absent otoacoustic emissions, with risk increasing more than six fold as the emission amplitude decreases (see, Lapsley et al., J. Acoust. Soc. Amer., 120(1):280-296 2006). The problem in applying these findings has been that the test-retest variability of objective OAE measurements is often so large as to make it difficult to detect the warning signs in individual cases, particularly at high-frequencies where changes in OAEs due to aging, noise exposure, and ototoxic drug use are expected to occur first.

OAEs can be measured with a low-noise microphone placed in the ear canal, either in the absence of any stimulation (spontaneous OAEs) or in response to acoustic stimulation (evoked OAEs). Because their measurement is noninvasive, evoked OAEs are particularly useful for assessing inner-ear function in humans, e.g., in newborn hearing screening programs or in patients at risk for developing a sensory hearing loss, e.g., due to work (e.g., construction, manufacturing, agriculture, mining, disc jockey, and rock musician), combat duty, or age.

Due to their dimensions, commonly used OAE measurement probe assemblies are coupled to the ear canal near its entrance, which alters the acoustic load impedance seen from the eardrum and also gives rise to acoustic standing waves that can affect both the measured OAE as well as the stimulus pressure that is used to evoke the OAE. For instance, the pressure level of the spontaneous OAE measured in a closed ear canal can be 10-15 dB higher at frequencies below 2 kHz as compared to open-canal measurements (Boul et al., “Spontaneous otoacoustic emissions measured using an open ear-canal recording technique,” Hear. Res., 269:112-121 (2010)).

While closed ear canal measurements are preferred in most settings, simply a change in the residual ear canal volume (i.e., the space between the OAE probe tip and the eardrum) can result in OAE level variations of about 2-3 dB at low frequencies for measurements obtained in the same ear, with extreme cases reporting changes of as much as 8 dB for evoked OAE frequencies near 5 kHz even when the evoking-stimulus level is corrected for the ear-canal acoustics (Scheperle et al., “Influence of in situ, sound-level calibration on distortion-product otoacoustic emission variability,” J. Acoust. Soc. Am., 124:288-300 (2008)).

### SUMMARY

The present disclosure provides two methods for accounting for the ear-canal acoustics on measured OAE pressure. Specifically, the new methods correct for the effects of the acoustic load on the measured OAE pressure and offer new metrics for displaying OAE results. The present disclosure enables one to represent the OAE pressure measured at the entrance of the ear canal as either the OAE pressure at the eardrum as it would appear in an anechoic ear canal (emitted pressure level, P_{EPL}) or as a Thévenin-equivalent OAE source pressure level at the eardrum (P_{TPL}). Either method can be used to correct the OAE pressure for the combined effects of the acoustics of the ear canal and OAE probe assembly. The present disclosure results in better test-retest repeatability as well as improved reliability of OAE measurements, which in turn would lead to better clinical sensitivity and specificity of the OAE tests.

As described herein, the methods have been applied to measurements obtained in human ear canals, but the new methods can also be applied to the ear canals of other animals and in any tube-shaped acoustic cavity so long as the load reflectance, the probe-system reflectance, and the one-way tube delay can be determined. As noted, the equations described herein use load reflectance and probe-system reflectance as parameters, however reflectance is closely related to absorbance and impedance and thus the equations herein can be easily rewritten using the load and probe-system absorbance or impedance as well.

The first method includes calculating the complex-valued OAE pressure emitted (P_{EPL}) at the eardrum as it would be measured if the eardrum were loaded with an anechoic tube of the same characteristic impedance as the ear canal. Because there are no reflections in an anechoic ear canal, P_{EPL} is not influenced by standing waves. This method for correcting the OAE pressure level is particularly useful when repeated measurements in the same ear are performed, such as in monitoring inner-ear health with OAEs in patients undergoing treatment with ototoxic drugs or who are routinely exposed to noise, e.g., through their occupation or as soldiers in a battlefield.

The second method for correcting the OAE pressure derives the Thévenin-equivalent OAE source pressure at the eardrum (P_{TPL}). The complex-valued pressure P_{TPL} corresponds to the OAE pressure measured in an acoustic open-circuit condition, when no external acoustic load is applied at the eardrum. Thus, P_{TPL} provides a measure of the OAE pressure at the eardrum that is completely load-independent and is not affected by standing waves. As compared to P_{EPL}, this approach may be favored when comparing emissions measured in ears with different characteristic impedances (i.e., cross sectional areas). This could be of relevance when, e.g., comparing OAE measured in adult and infant ears, whose ear canals are considerably smaller.

In certain embodiments of the present disclosure, the measurements are performed with an OAE probe that contains a microphone and a sound source, coupled to the ear canal with a rubber/foam tip. The sound source is used to generate a calibration stimulus used in measurements of the acoustic properties of the ear canal that are necessary for calculating P_{EPL} and P_{TPL}. If evoked OAEs are measured, the sound source is used to generate the evoking stimulus (e.g., one, two, or more tones). In such a case, it must be assured that the evoking stimulus has been calibrated with a method that corrects for the ear-canal acoustics. Otherwise, the OAE expressed using either of the new methods (metrics) would reflect the effects of ear-canal acoustics on the evoking stimulus, thus yielding an OAE pressure level that still depends on the specific configuration of the measurements.

Both of the new methods described herein for compensating the OAE pressure for the ear canal acoustics rely on the ability to accurately measure in situ the reflectance/absorbance/impedance of the OAE probe and the ear canal as well as the ear-canal one-way delay. Such measurements can be performed with various known techniques, e.g., as described by (Keefe et al., “Ear-canal impedance and reflection coefficient in human infants and adults,” J. Acoust. Soc. Am., 94:2617-2638 (1993)).

In one aspect, the disclosure provides methods for measuring OAEs in a subject, such as a human infant or adult, or an animal, such as a cat, dog, monkey, chimpanzee, rodent, or other domesticated animal, using an OAE probe, wherein the measurement is corrected for the subject's ear canal acoustics and for the OAE probe. The methods include (a) inserting the OAE probe into the subject's ear canal; (b) delivering a calibration stimulus into the ear canal with the OAE probe and detecting any calibration signal propagated from within the ear canal; (c) using the detected calibration signal to calculate calibration measurements comprising ear canal reflectance, ear canal one-way delay, and OAE probe reflectance; (d) delivering an excitation stimulus sufficient to evoke an OAE into the ear canal with the OAE probe; (e) collecting any OAE response; (f) converting the OAE response using the calculated calibration measurements from step (c) into an unbiased OAE response; and (g) displaying the unbiased OAE response.

In some implementations of these methods the calibration signal can be further used to calibrate the excitation stimulus used to evoke the OAE. In some embodiments, the excitation stimulus is a wide-band chirp that covers the range of frequencies within the human audible range. In some implementations, the step of detecting any calibration signal emitted from within the ear canal includes of consists of detecting a pressure from within the ear canal. In certain implementations, the step of converting the OAE response includes correcting OAE amplitude and phase. For example, correcting OAE amplitude and phase can include calculating emitted pressure (P_{EPL}) or Thévenin-equivalent source pressure (P_{TPL}) using the calibration measurements.

In some implementations, the OAE response measured at the OAE probe (P_{SPL}) is converted to emitted pressure (P_{EPL}) using the equation:

$${P}_{EPL} = {P}_{SPL} \frac{( 1 - {R}_{EC} {R}_{s} )}{t ( 1 + {R}_{s} )}$$

where R_{EC} is the ear-canal reflectance, R_{S} is the OAE probe reflectance, and t is equal to e^{-2πfτ}, with τ corresponding to one-way ear canal delay. In other implementations, the OAE response measured at a microphone in the OAE probe (P_{SPL}) is converted to Thévenin-equivalent source pressure (P_{TPL}) using the equation:

$${P}_{TPL} = {P}_{SPL} \frac{2 t ( 1 - {R}_{EC} {R}_{s} )}{( 1 + {R}_{s} ) ( {t}^{2} - {R}_{EC} )}$$

where R_{EC} is the ear-canal reflectance, R_{S} is the OAE probe reflectance, and t is equal to e^{-2πfτ}, with τ corresponding to one-way ear canal delay.

Any of the new methods can further include using the displayed unbiased OAE response to determine the health of the inner ear of the subject, e.g., using known techniques.

In another aspect, the disclosure provides methods for calculating complex otoacoustic emission (OAE) emitted sound pressure (P_{EPL}) at the eardrum, equivalent to a complex OAE pressure measured in an anechoic ear canal. These methods include: (a) measuring the complex OAE sound pressure (P_{SPL}) with an OAE probe microphone coupled to the ear canal; (b) measuring the ear canal reflectance (R_{EC}), OAE probe reflectance (R_{S}), and one-way ear canal delay (τ) using the same probe position used in the P_{SPL} measurements; and (c) at any frequency f calculating the P_{EPL} according to:

$${P}_{EPL} = {P}_{SPL} \frac{( 1 - {R}_{EC} {R}_{s} )}{t ( 1 + {R}_{s} )} ,$$

where t=e^{-2πfτ}.

In another aspect, the disclosure provides methods for calculating a load-independent Thévenin-equivalent complex OAE source pressure at the eardrum (P_{TPL}). These methods include: (a) measuring the complex OAE sound pressure (P_{SPL}) with an OAE probe microphone coupled to the ear canal; (b) measuring the ear canal reflectance (R_{EC}), OAE probe reflectance (R_{S}), and one-way ear canal delay (τ) using the same probe position used in the P_{SPL} measurements; and (c) at any frequency f calculating the P_{TLP} according to:

$${P}_{TPL} = {P}_{SPL} \frac{2 t ( 1 - {R}_{EC} {R}_{s} )}{( 1 + {R}_{s} ) ( {t}^{2} - {R}_{EC} )} ,$$

where t=e^{-2πfτ}.

In any of the methods described herein, a preliminary step may include calibrating the OAE probe itself in a set of dummy loads before inserting the OAE probe into the subject's ear.

As used herein, the characteristic impedance of the ear canal (Z₀) is defined as:

$${Z}_{0} = \frac{ρ c}{A}$$

where ρ is the density of the air, c is the velocity of sound in air, A is the cross sectional area of the canal.

As used herein, ear canal pressure reflectance (R_{EC}) is defined as:

$${R}_{EC} = \frac{{Z}_{EC} - {Z}_{0}}{{Z}_{EC} + {Z}_{0}}$$

where Z_{EC} is the complex-valued ear canal acoustic impedance and Z₀ is the characteristic impedance of the ear canal.

As used herein, OAE source pressure reflectance (R_{S}) is defined as:

$${R}_{S} = \frac{{Z}_{S} - {Z}_{0}}{{Z}_{S} + {Z}_{0}}$$

where Z_{S} is the Thévenin-equivalent complex-valued source impedance and Z₀ is the characteristic impedance of the ear canal.

As used herein, one-way ear canal delay is defined as:

$$τ = \frac{1}{2 {f}_{λ \text{/} 2}}$$

where f_{λ/2} is the first half-wave resonance frequency of the ear canal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated herein by reference in their entirety. For example, the present disclosure incorporates by reference all of the subject matter and figures disclosed in Karolina K. Charaziak and Christopher A. Shera, “Compensating for Ear-Canal Acoustics when Measuring Otoacoustic Emissions,” J. Acoust. Soc. Am., 141(1): 515-531 (January 2017).

In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a graph of an example of conventional distortion product OAE (DPOAE) measurements obtained in a human subject using an OAE probe placed at two positions in the ear canal (shallow—position 1, black line; deep—position 2, red dotted line).

FIG. 1B is a graph based on the data in FIG. 1A showing the difference in the DPOAE levels obtained at the two probe positions (position 2 minus position 1, black dashed line) and when the measurements were repeated (retest) at the same position (1)(gray line).

FIG. 2A is a schematic diagram of a model of a microphone and sound source in a cavity.

FIG. 2B is a graph of the magnitude of the relationship between P_{SPL} (OAE sound pressure level), P_{EPL} (OAE pressure at the eardrum as measured in an anechoic ear canal (emitted pressure level)), and P_{TPL} (Thévenin-equivalent OAE source pressure at the eardrum) over different frequencies) measured in a setup depicted in FIG. 2A with the sound source serving as an OAE source.

FIG. 3 is a flowchart for an implementation of a method as described herein for measuring the OAEs unbiased by ear-canal acoustics.

FIG. 4A is a schematic diagram of a sound source (e.g., a speaker) placed in an anechoic (long, about 50 feet) brass tube (inner diameter of 7.9 mm). In this model, P_{EPL} was directly measured with a probe microphone (ERIC).

FIG. 4B is a graph of the results of tests in the models of FIGS. 4A and 4C and shows the difference in level (dB) vs. frequency of directly measured (model in FIG. 4A) and calculated (from model in FIG. 4C) P_{EPL} for six measurements (grey lines, the mean is shown by the black line).

FIG. 4C is a schematic diagram of a sound source (e.g., a speaker) placed in a short brass tube (about 20 mm). In this model, P_{SPL} pressure was measured with an OAE probe (ER10X), as done in human ears, and converted to P_{EPL}.

FIG. 4D is a graph of the results of tests in the models of FIGS. 4A and 4C and shows the difference in phase slope (group delay)(ms) vs. frequency of directly measured (model in FIG. 4A) and calculated (from model in FIG. 4C) P_{EPL} for six measurements (grey lines, the mean is shown by the in black line).

FIGS. 5A to 5D are a series of graphs showing the results of DPOAE measurements obtained from one human subject using shallow probe placement. FIGS. 5A and 5C show the functions magnitude (5A) and phase (5C) used to convert DPOAE P_{SPL} to either P_{EPL} (black line) or to P_{TPL} (red line). FIGS. 5B and 5D show the DPOAE level and phase before the conversion (P_{SPL}, dashed black line) and after (P_{EPL}, solid red line and P_{TPL} in dashed red line). Noise is shown in grey.

FIGS. 6A to 6F show the sensitivity of different OAE metrics to the change in the residual acoustic space created by varying the position of the OAE probe relative to the OAE source.

FIG. 6A is a schematic diagram of a model of an OAE probe inserted into one end of a cavity (at positions 1 or 2) that approximates the dimensions of the human ear canal and a sound source (speaker) located at the other end of the cavity.

FIG. 6B is graph of the results of tests in the model of FIG. 6A showing the change in level (dB) due to moving the OAE probe from position 1 to 2 at different frequencies.

FIG. 6C is a graph of the results of tests in the model of FIG. 6A showing the change in phase slope (group delay)(ms) due to moving the OAE probe from position 1 to 2 at different frequencies.

FIG. 6D is a schematic diagram of a human ear in cross-section showing an OAE probe inserted into the outer ear canal at positions 1 and 2 with the cochlea representing the OAE source. OAEs were evoked using a pair of tones (DPOAE).

FIG. 6E is a graph of the results of tests in the human ear shown in FIG. 6D showing the change in level (dB) due to moving the OAE probe from position 1 to 2 at different DPOAE frequencies.

FIG. 6F is a graph of the results of tests in the human ear shown in FIG. 6D showing the change in phase slope (group delay)(ms) due to moving the OAE probe from position 1 to 2 at different DPOAE frequencies.

FIG. 7 is a graph of the mean change (+/−SEM) in DPOAE levels expressed using different metrics due to a change in the probe position in the ear canal (deep minus shallow) for n=5 human subjects.

FIGS. 8A and 8B are graphs that show stimulus-frequency OAEs (SFOAEs) using a single tone measured in one ear at shallow (black) and deep (red) probe positions both before (FIG. 8A) and after (FIG. 8B) conversion to emitted pressure level (EPL). Data segments with SNR<6 dB are shown using dotted lines.

### DETAILED DESCRIPTION

The present disclosure provides two methods of accounting (e.g., correcting) for the confounding effects of acoustic load on the measurements of otoacoustic emissions (OAEs). Such effects have been shown to influence the measured OAE pressure with the OAE probe microphone (P_{SPL}) at OAE frequencies <5 kHz (e.g., Scheperle et al., 2008, supra), but as described herein even larger effects were observed at frequencies above about 5 kHz, e.g., above 6 or 7 kHz. The acoustic load can change, for example, by changing the distance (L) between the OAE probe and the eardrum, which shifts the half wave-resonant frequency of the ear canal (f_{λ/2}), which leads to variation in the OAE pressure of 10-15 dB at these higher frequencies. In human ear canals, the OAE probe is typically placed 18-24 mm away from the eardrum, thus the effects of the half-wave resonant frequency on the OAE pressure is significant for frequencies of 5 kHz and higher, depending on the exact placement of the probe in the ear canal ((f_{λ/2}·0.5c/L, where c is the speed of sound). Because changes of as little as 3-6 dB in OAE levels are typically considered clinically meaningful, it is clear that with no correction for the effects of ear-canal acoustics on the OAE pressure level, the rate of erroneous test results can be exacerbated.

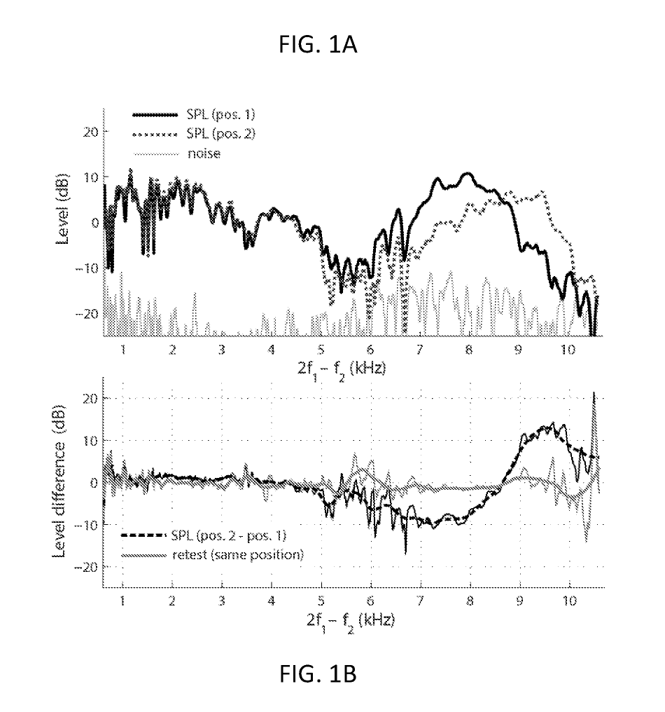

FIG. 1A shows the results of one example in which OAEs were measured using an Etymotic Research ER10X OAE probe system in response to stimulation with two tones (with tone levels L₁, L₂ of 62.52 dB forward pressure level (FPL), at a fixed frequency ratio, f₂/f₁, of 1.22 with f₂ swept from 1 to 16 kHz). The resulting OAE, the so-called distortion product (DP) OAE (DPOAE), was measured at frequency of 2f₁−f₂. The tests were done without any of the corrections using the new methods described herein. The measurements were obtained for a shallow-probe insertion (FIG. 1A, black line) and deep probe insertion (FIG. 1A, dashed red line) for assessing the change in DPOAE level with probe position (bottom panel, red). In FIG. 1A, the noise floor is shown in gray. As shown in FIG. 1B, the DPOAEs were re-measured for the shallow probe placement to assess the test-retest repeatability unrelated to the probe placement (grey line in FIG. 1B). DPOAE levels were expressed in a conventional way (SPL—black dashed line in FIG. 1B).

The change in the DPOAE level due to changing the probe position (i.e., intentional change in the acoustic load impedance) is shown in the graph in FIG. 1B (black) and compared to DPOAE test-retest repeatability where the measurement was repeated for unchanged probe position (grey). It is clear that the effects of the ear canal acoustics on OAE pressure are particularly large at frequencies above 5 kHz and could be misinterpreted as a clinically significant change in inner ear health. While small changes in P_{SPL} at lower frequencies relate to the change in the volume of the ear canal space created by pushing the OAE probe forward towards the eardrum, the changes at higher frequencies (>5 kHz) appear to relate to the problem of standing waves, e.g., a change in f_{λ/2}. Thus, to interpret the OAE as an indicator of the inner ear health the measured OAE must be corrected for the effects of the acoustic load.

### Methods of Correcting for the Effects of Acoustic Load on OAE Measurements

The new methods described herein correct for this significant problem. To begin, one needs first to measure the ear canal reflectance (R_{EC}), the OAE probe source reflectance (R_{S}), and the one-way ear canal delay (τ). As noted above, the equations described herein use load (ear canal) reflectance and probe-system (probe source) reflectance as parameters, however reflectance is closely related to absorbance and impedance and thus the equations herein can be easily rewritten using the load and probe-system absorbance or impedance as well.

In general, the first method includes calculating the OAE pressure emitted (P_{EPL}) at the eardrum as it would be measured if the eardrum were loaded with an anechoic tube of the same characteristic impedance as the canal. Because in an anechoic ear canal there are no reflections, P_{EPL} is not influenced by standing waves. This method for correcting the OAE pressure level is particularly useful when repeated measurements in the same ear are performed, such as in monitoring the inner-ear health with OAEs in patients undergoing treatment with ototoxic drugs, older patients, and patients who are routinely exposed to noise, e.g., through their occupation, e.g., construction, manufacturing, agriculture, mining, disc jockey, rock musician, or combat duty.

In general, the second method for correcting the OAE pressure derives the Thévenin-equivalent OAE source pressure at the eardrum (P_{TPL}). The P_{TPL} corresponds to the OAE pressure measured in an acoustic open-circuit condition, when no external acoustic load is applied at the eardrum. Thus, P_{TPL} provides a measure of the OAE pressure at the eardrum that is completely load-independent and is not affected by the standing waves. As compared to P_{EPL}, this second approach may be favored when comparing emissions measured in ears with different characteristic impedances (i.e., cross sectional areas). This could be of relevance when, e.g., comparing OAE measured in adult and infant ears, whose ear canals are considerably smaller, or as an infant or child grows over time.

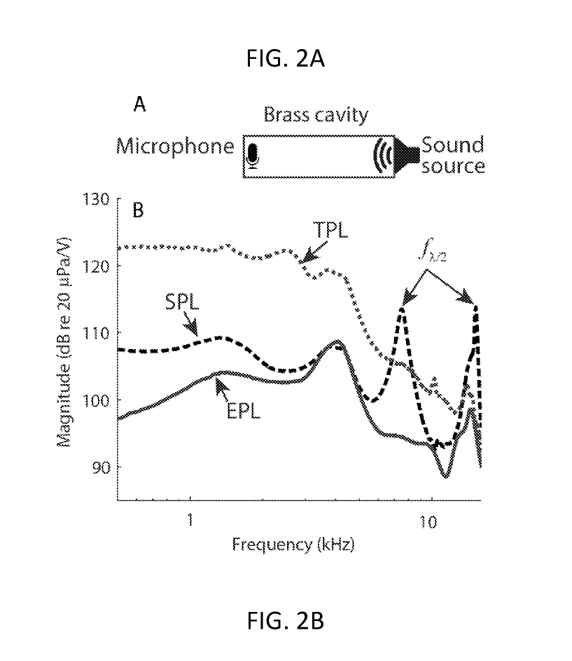

The relationships between P_{SPL}, P_{EPL} and P_{TPL} were demonstrated in a model consisting of a brass tube (an analog of the ear canal) and a speaker (an analog of OAE source at the eardrum, see FIG. 2A for schematic representation of the measurement system) where sound produced by the speaker can be tightly controlled (unlike the real OAE in the ear). The test results are shown in the graph of FIG. 2B. P_{EPL} and P_{TPL} were derived using independent methods. P_{EPL} was measured in an anechoic tube of the same diameter as tube depicted in FIG. 2A and P_{TPL} was derived from Thévenin-equivalent source calibration procedure, e.g., (Scheperle et al., 2008). Both P_{EPL} (solid red) and P_{TPL} (dotted red) were compared to predicted P_{SPL} at the terminal end of a brass tube (the diameter and the length of the tube was chosen to approximate an adult human ear canal) (dashed black, FIG. 2B). In this setting, one may consider the source of the emission as characterized by its Thévenin-equivalent P_{TLP}, P_{SPL} the pressure measured with an OAE probe microphone when the OAE source is loaded with a tube terminated by the OAE probe and P_{EPL} the OAE pressure as measured at the eardrum when OAE source is loaded with an anechoic tube with characteristic impedance Z₀.

When the sound source is loaded with a tube of a length L terminated at the other end with OAE probe, the reflections within the enclosed space give a rise to standing waves. When the sound pressure is measured near the termination of the tube with a microphone (P_{SPL}—as usually done for measurements of OAEs), a decrease in pressure as compared to P_{TPL} is observed at low frequencies (due to the load impedance) and an increase in the pressure response is shown near frequencies of the half-wave resonance (f_{λ/2})—the frequency f_{λ/2} is determined by the length L of the tube. In contrast, neither P_{TPL} nor P_{EPL} are influenced by the standing wave at f_{λ/2} and provide unbiased by ear-canal acoustics metrics of OAE pressure. A more detailed description of the new methods follows.

To account for and correct for the effects of the acoustic load on the OAE signal, the ear canal was modeled as a simple tube using a generic two-port system with port #1 representing the eardrum and port #2 representing the OAE probe microphone. The system, driven by a Thévenin-equivalent source pressure, was described using a scattering matrix for a special case of a simple tube (Shera & Zweig, 1992). The scattering matrix relates the forward and reverse traveling pressure waves at each port. In this model, the initial outgoing wave at port #1 is equivalent to initial outgoing OAE wave at the eardrum, referred here as emitted pressure (P_{EPL}) such as:

$${P}_{EPL} = {P}_{SPL} \frac{( 1 - {R}_{EC} {R}_{s} )}{t ( 1 + {R}_{s} )}$$

where P_{EPL} is the complex emitted pressure at frequency f, P_{SPL} is the complex OAE pressure at frequency f measured with the OAE probe microphone; R_{EC} and R_{S} are, respectively, the ear-canal and OAE-probe source reflectances at frequency f, and t is equal to e^{-2πfτ} with τ corresponding to one-way ear canal delay. The complex pressure P_{EPL} is equivalent to the OAE pressure as measured at the eardrum in an anechoic ear canal with the same characteristic impedance. Thus, unlike P_{SPL}, P_{EPL} does not depend on the acoustics of the residual ear-canal space. If it is desired to quantify the OAE using acoustic power rather than pressure, the emitted OAE intensity is given by:

$${I}_{EIL} = \frac{{\vert {P}_{EPL} \vert}^{2}}{2 {Z}_{0}}$$

where, P_{EPL} is the complex OAE emitted pressure and Z₀ is characteristic impedance of the ear canal.

The two-port model described by a scattering matrix allows also to express the complex Thévenin-equivalent sound-pressure (P_{TPL}) in terms of the total complex sound-pressure at port #2 (at the microphone, P_{SPL}) at any given frequency f as:

$${P}_{TPL} = {P}_{SPL} \frac{2 t ( 1 - {R}_{EC} {R}_{s} )}{( 1 + {R}_{s} ) ( {t}^{2} - {R}_{EC} )}$$

The pressure P_{TPL} correspond to the OAE pressure as measured in an acoustic open circuit; thus it is completely independent of the acoustic load imposed at the eardrum.
The two pressures P_{TPL} and P_{EPL} are related as:

$${P}_{TPL} = {P}_{EPL} \frac{2}{( 1 - {R}_{EC} \text{/} {t}^{2} )}$$

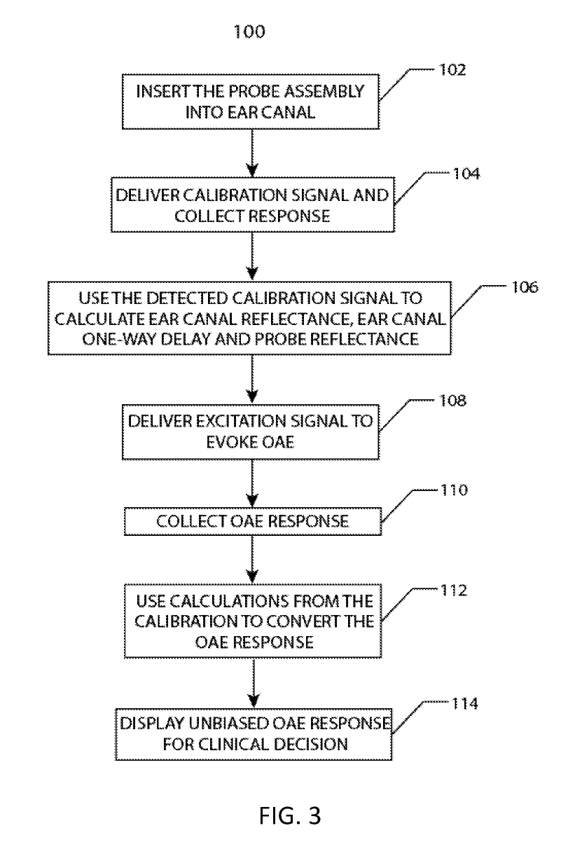

FIG. 3 is an example of flowchart for application of the two methods to OAE measurements. These steps are carried out for each patient for each new OAE measurement. Method 100 includes a process 102 for placing the OAE probe assembly into the ear canal, a process 104 for measuring the pressure generated in the ear canal in response to a calibration stimulus, a process 106 for calculating ear-canal reflectance (R_{EC}), OAE-probe reflectance (R_{S}) and one-way ear canal delay (τ), a process 108 for delivering a stimulus evoking OAEs (e.g., one, two, or more stimulus tones), a process 110 for collecting the OAE with a probe microphone (P_{SPL}) in the ear canal, a process 112 for correcting the OAE amplitude and phase (i.e., calculating P_{EPL} or P_{TPL}) for the acoustic load parameters derived from calibration measurements, a process 114 for displaying the OAE measurements using the corrected metrics for interpretation of the inner ear health. Although the above method has been described using a specific sequence of processes, there can be many alternatives and modifications. For instance, for measurements of spontaneous OAEs the process 108 can be completely omitted. The order of the processes can be changed as well, so long as all the measurements (calibration and OAEs) are obtained for the unchanged OAE probe position and configuration.

At the process 104 a stimulus is delivered to the ear canal using a sound source transducer positioned at the entrance of the ear canal. In one embodiment of this disclosure, the sound source is a part of the OAE probe assembly, such as in an Etymotic Research ER10X probe. The choice of the calibrating stimulus is up to the investigator, so long as it covers the frequency range of the subsequent OAE measurements. In the present embodiment, a useful stimulus is a wide-band chirp that covers the range of frequencies within the human audible range. The calibration stimulus level should be chosen so that it is low enough to avoid evoking the contraction of the middle-ear muscles, but high enough that the measured pressure level is dominated by the passive reflections within the ear canal rather than by the OAE pressure generated in the cochlea. In most cases, the calibration levels of 50-60 dB SPL meet these criteria.

In some implementations a preliminary step may be required to calibrate the OAE probe assembly itself in a set of dummy loads using standard techniques before inserting the OAE probe into the subject's ear.

At the process 106 the measured ear-canal responses to a calibration stimulus are used to calculate the values of R_{EC}, R_{S}, and τ. There are multiple ways to derive and obtain these quantities in situ, some of which are detailed in (Keefe, 1998, supra). In one embodiment, the values of R_{EC} and R_{S} are calculated using prior knowledge of the OAE probe Thévenin-equivalent source impedance and pressure derived from a separate calibration measurements obtained in a set of acoustic loads of known impedances. This approach is detailed in (Scheperle et al., 2008, supra). The one-way ear-canal delay may be obtained using measurements of time-domain reflectance as described in (Rasetshwane & Neely, 2011) or from the frequency of the first half-wave resonance (e.g., measurements are detailed in Souza et al., “Comparison of nine methods to estimate ear-canal stimulus levels,” J. Acoust. Soc. Am., 136:1768-178 (2014)) as used in the embodiment detailed here. Although processes 104 and 106 could be completed after the processes 108 and 110, it is recommended to keep the order exemplified in FIG. 3 for at least two reasons.

First, the detected calibration signal can be helpful in evaluating the OAE probe fit in the ear canal as described in (Groon et al., “Air-leak effects on ear-canal acoustic absorbance,” Ear Hear., 36:155-163 (2015)). Second, the calibration signal can be used for calibrating the stimulus used to evoke OAEs in process 108. To measure an evoked OAE that is fully independent of the acoustic load imposed by the ear canal and OAE probe assembly it is important to calibrate the evoking stimulus with a method that eliminates the effects of standing waves on the stimulus. In the present embodiment and all examples of measurements obtained in human ears, the stimulus was calibrated using a forward-pressure level (FPL) calibration method as detailed in (Scheperle et al., 2008, supra). Alternative stimulus calibration methods are described in (Souza et al., 2014, supra).

At the process 110, the OAE response is acquired with the OAE probe microphone. Depending on the type of the OAE, different measurements and averaging techniques can be used here. In the examples described below, distortion-product (DP) OAEs were measured in response to two tones swept across wide range of frequencies at moderate levels.

At the process 112, the OAE measured at the microphone (P_{SPL}) is converted to either emitted pressure (P_{EPL}) following the equation:

$${P}_{EPL} = {P}_{SPL} \frac{( 1 - {R}_{EC} {R}_{s} )}{t ( 1 + {R}_{s} )}$$

or to Thévenin-equivalent source pressure (P_{TPL}) following the equation:

$${P}_{TPL} = {P}_{SPL} \frac{2 t ( 1 - {R}_{EC} {R}_{s} )}{( 1 + {R}_{s} ) ( {t}^{2} - {R}_{EC} )}$$

where R_{EC} is the ear-canal reflectance, R_{S} is the OAE probe reflectance, and t is equal to e^{-2πfτ}, with τ corresponding to one-way ear canal delay.

Step 114 is to display the unbiased OAE response, now corrected for the confounding effects of acoustic load on the OAE measurements. The display can be used by the operator or clinician to make a clinical decision.

### EXAMPLES

The new methods are further described in the following examples, which do not limit the scope of the invention described in the claims.

### Instrumentation

In the methods described herein, stimulus waveforms were generated and responses acquired and averaged digitally at a sampling rate of 48 kHz using a RME Babyface® Audio Interface (Audio AG, Haimhausen, Germany) and an ER10X OAE probe system (Etymōtic Research, Elk Grove Village, Ill.). A custom written software written in MATLAB® (The Mathworks, Natick, Mass.) was used to control the hardware and analyze the data as described herein. This software is based on the equations and method steps described herein and causes the system to carry out the steps in flow chart of FIG. 3. The microphone signal was amplified (20 dB), high-pass-filtered (cutoff frequency of 100 Hz) and corrected for the microphone sensitivity (Siegel, “Calibration of otoacoustic emission probes. In: Otoacoustic Emissions: Clinical Applications, Third Edition (Robinette et al., eds.), pp 403-429 (New York: Thieme, 2007)). Thévenin-equivalent probe parameters (source pressure, P_{S}, and impedance, Z_{S}) were measured daily at room temperature using constant attenuation chirp-responses measured in an ER10X calibrator brass-tube (i.d., 7.9 mm) for five different length settings (70, 62, 54, 37 and 28 mm) for each sound source separately (see Scheperle et al., 2008, supra for details; Souza et al., 2014, supra). The measurements were repeated until the so-called “calibration error” (calculated over 2-8 kHz range) was less than 1 (typically ˜0.03).

All measurements were performed in a sound-isolated chamber. Before each OAE test, wide-band chirp responses were collected in the ear canal. These responses were used to: a) estimate the first half-wave resonant frequency, f_{λ/2}, b) judge the probe seal, c) calibrate the DPOAE stimuli in situ, and d) derive the pressure reflectance of the OAE probe and the ear canal for P_{EPL} and P_{TPL} calculations.

The accurate measurement of the f_{λ/2} was facilitated by normalizing ear canal chirp response by the chirp response obtained beforehand in a 50-ft long coil of copper tube (i.d.=7.9 mm; Souza et al., 2014 supra). This normalization removes most of the irregularities of sound sources frequency response that could obscure the assessment of f_{λ/2}. The half-wave resonant frequency f_{λ/2} was used to estimate τ one-way ear canal delay. The probe was considered sealed to the ear canal when the low-frequency ear-canal absorbance was ≤0.29 and the low-frequency admittance angle was >44° (averaged over 0.2-0.5 kHz, adapted from Groon et al., 2015, supra).

### Example 1—Simulated OAE Measurements in a Cavity

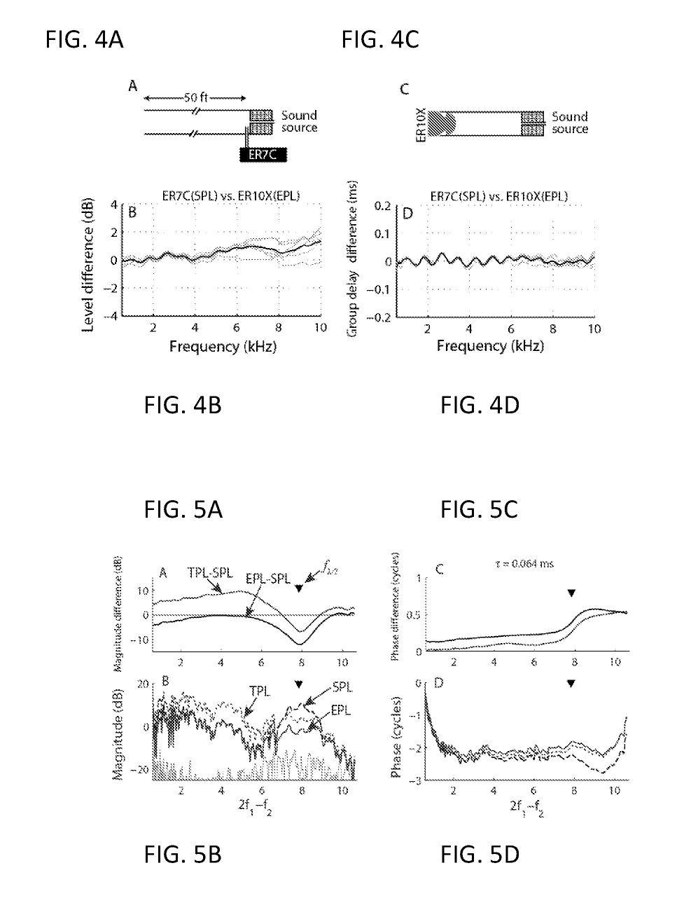

As P_{EPL} represents the source pressure measure in an anechoic cavity, the calculation shown above can be verified by comparing the calculations to direct measurements. Such measurements cannot be obtained in human ears (as anechoic ear canals do not exist), but we employed a simple measurement system consisting of an anechoic tube and closed tube terminated with a sound source (a modified Audax, TW010F1, coupled via plastic tubing to a foam tip sealed to the end of the tube) that served as an equivalent of the OAE source pressure at the eardrum (see FIGS. 4A and C).

The sound source was driven by a constant-voltage chirp stimulus (˜50 dB SPL). The dimensions of the closed tube (i.d.=7.9 mm, L=30 mm) were chosen to approximate the dimensions of an adult ear canal. When the P_{EPL} measured near the sound source directly with a small probe microphone (ERIC, FIG. 4A) is compared to the P_{EPL} extracted from the P_{SPL} measurement with OAE probe (ER10X, FIG. 4C) obtained in the closed tube the magnitude and phase-gradient group delays agree within +/−2 dB and +/−0.03 ms (FIGS. 4B and 4D, respectively, in black—mean, in grey—individual measurements, n=6), which is close to test-retest repeatability of such measurements.

### Example 2—OAE Measurements in Human Subjects

Subjects were five normal-hearing young adults (22-30 years old, 2 males), all with audiometric thresholds <15 dB hearing level (HL) for frequencies 0.5 to 16 kHz (Lee et al., 2012), no history of ear disease and normal results of otoscopic examination. The ear that emitted higher levels of DPOAEs at high-frequencies was chosen for testing (six right ears and two left ears).

DPOAEs were recorded at 2f₁−f₂ (0.6-10.6 kHz) with primary tone levels L₁, L₂ of 62, 52 dB (dB FPL) at a fixed primary frequency ratio, f₂/f₁, of 1.22. The primary frequencies were swept upward logarithmically at rate of 1 octave/sec (Long et al., “Measuring distortion product otoacoustic emissions using continuously sweeping primaries,” J. Acoust. Soc. Am., 124:1613-1626 (2008); Abdala et al., “Optimizing swept-tone protocols for recording distortion-product otoacoustic emissions in adults and newborns,” J. Acoust. Soc. Am., 138:3785-3799 (2015)). The stimuli were calibrated to produce a constant forward pressure level in the ear canal (Scheperle et al., 2008).

The range of tested frequencies was divided into three sweeps (each lasting 1.43 sec), so that within each sweep f₂ changed from 0.96, 2.4 and 6.1 kHz to 2.6, 6.6 and 16.5 kHz, respectively, resulting in 0.1 octave overlap between start/stop frequencies. To facilitate data collection the three primaries sweeps were presented concurrently. Fast data collection was important here to minimize any changes in DPOAE levels due to probe slippage, inherit changes in OAE over time etc. Data collection was stopped after accumulating 96 artifact-free averages (see Kalluri and Shera, “Measuring stimulus-frequency otoacoustic emissions using swept tones,” J. Acoust. Soc. Am., 134:356-368 (2013) for a description of a real-time artifact rejection algorithm for swept-tone OAEs). Phase-rotation averaging was employed to cancel out the f₁ and f₂ primaries from the measured response (Whitehead et al., “Visualization of the onset of distortion-product otoacoustic emissions, and measurement of their latency,” J. Acoust. Soc. Am., 100:1663-1679 (1996)).

A non-FFT based analyses, Least Squares Fit (LSF) technique, was used to estimate DPOAE amplitude and phase (Long et al., 2008, supra). In this LSF technique, the models of DPOAE and primary tones are fitted to the signals recorded in the ear canal by minimizing the sum of squared residuals between the model and the data. The LSF was conducted on short chunks of overlapping Hann-widowed data with specified duration. The window duration must be adjusted to account for the sweep rate and to accommodate the frequency-dependent latency shifts in the so called reflection component of the total DPOAE (Shera and Guinan, “Evoked otoacoustic emissions arise by two fundamentally different mechanisms: a taxonomy for mammalian OAEs,” J. Acoust. Soc. Am., 105:782-798 (1999).

Prior to unwrapping, DPOAE phase at 2f₁−f₂ was corrected for phase variation of the primaries by subtracting 2ϕ₁−ϕ₂, where ϕ₁, ϕ₂ are the phases of the either forward pressure at the frequencies of f₁ and f₂. The group delay was calculated as a negative slope of the OAE phase vs. frequency. The noise floor was estimated by taking the difference between adjacent sweep pairs and applying the LSF to this difference trace. Note that any possible confounding effects of our data collection and analysis methods are not crucial for interpretation of the results as we evaluated changes in DPOAEs with insertion depth obtained for different stimulus calibration conditions and OAE metrics, all obtained with the same sweep-tones and LSF routines.

The DPOAEs were measured for FPL-calibrated stimuli for the OAE probe sealed near the entrance of the ear canal (shallow insertion depth) and then the measurements were repeated for the probe pushed deeper into the ear canal by about 3 mm (deep insertion depth). The change in the probe position was judged based on the change in f_{λ/2}. The difference between DPOAE levels and phase-gradients group delays obtained for the two probe placements was our outcome measure. These differences were computed and compared between DPOAEs expressed as P_{SPL}, P_{EPL}, and P_{TPL}.

Following the measurements for deep probe placement, the probe was retracted back to the shallow placement, and another DPOAE response was obtained. Care was taken to match the f_{λ/2} to the f_{λ/2} obtained during the first “shallow” measurements. The difference in DPOAE levels and phase-gradients group delays for the two shallow probe placements (bracketing the deep-placement measurement) was taken as an estimate of DPOAE test-retest repeatability, and served as a reference for assessing the significance of the changes in DPOAEs obtained for deep and shallow placements. The DPOAE levels (P_{SPL}) near the f_{λ/2} met signal-to-noise criterion of at least 10 dB. This criterion was reinforced so the shifts in DPOAE levels near the f_{λ/2} could be reliably measured with changing the insertion depth.

An example of the conversion of P_{SPL} to either P_{TPL} or P_{EPL} is shown in FIGS. 5A to 5D for measurements obtained in a human subject. The differences between the three types of metrics are similar to ones observed in a cavity driven by a sound source (see FIG. 1B)—the P_{TPL} has larger magnitude than P_{SPL} as it represents the OAE pressure measured in load-free setting, with an exception of the peaks at the half-wave resonant frequencies, where standing waves obscure the P_{SPL}. P_{EPL} is lower in level than P_{SPL} at low-frequencies (because the acoustic load imposed by an anechoic tube is less than in a closed tube condition) and near the resonant frequencies (due to P_{SPL} being contaminated by standing waves). The upper panels show the magnitude (FIG. 5A) and phase (FIG. 5C) of the function used to convert P_{SPL} to P_{TPL} (red) and to P_{EPL} (black) derived from measurements obtained during the calibration procedure.

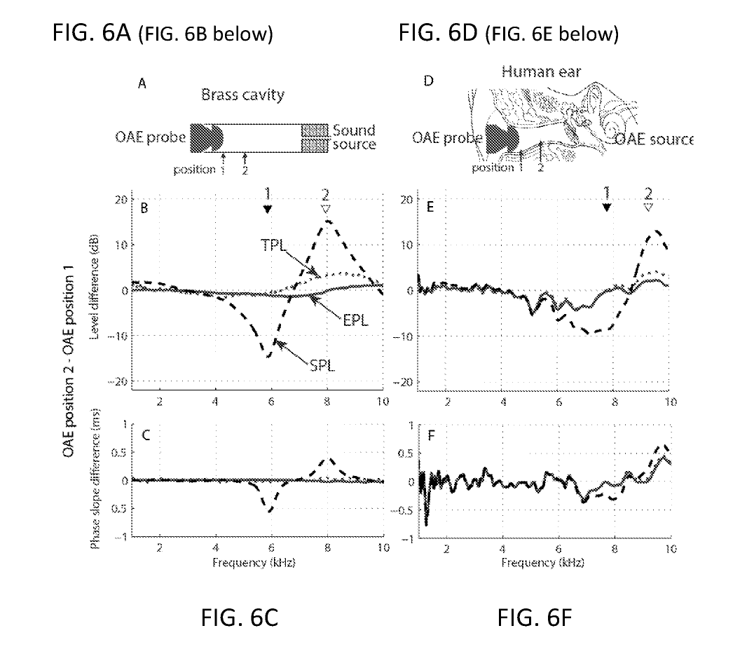

To illustrate the effectiveness of the new methods, the sensitivity of P_{EPL} and P_{TPL} to the changes in the acoustic load induced by shifting the position of the OAE probe relative to the sound source in a uniform brass tube (i.d.=7.9 mm) was tested. FIG. 6A shows a schematic of the measurement device and condition. The sound source was driven by a constant voltage while P_{SPL} was measured for two different positions of the OAE probe (marked as positions 1 and 2 in FIG. 6A). The change in P_{SPL} (FIG. 6B, black dashed) and phase slope (FIG. 6C, black dashed) due to changing the position of the OAE probe is striking, particularly near the half-wave resonant frequencies marked with triangles in FIG. 6B for each position. In contrast, when P_{SPL} obtained for each probe position was converted to either P_{EPL} (EPL, red solid) or P_{TPL} (TPL, red dotted) the sensitivity to probe position (i.e., the acoustics of the residual tube space) was nearly eliminated.

Analogous measurements were obtained in a human ear canal (FIG. 6D) for OAEs evoked with a pair of tones (DPOAEs) as described above. Because the two-tone stimulus was calibrated using a forward pressure calibration (Scheperle et al., 2008), allowing delivery of the stimulus that stimulates the cochlea uniformly independent of the probe position in the ear canal (Souza et al., 2014, supra), the change in DPOAE pressure (P_{SPL}, FIG. 6E and FIG. 6F, dashed black) as measured with the OAE probe microphone shifted from position 1 to 2 is assumed to be due to effects of the residual ear-canal acoustics on the OAE itself and not on the evoking stimulus. When the DPOAE pressure is converted to either P_{EPL} (solid red) or P_{TPL} (dotted red), the sensitivity to the OAE probe position is greatly diminished, particularly near the frequencies of the half-wave resonances (triangles).

The effectiveness of the P_{EPL} and P_{TPL} transformations depends heavily on the accuracy of the R_{S} and R_{EC} measurements. The estimation of the one-way ear canal delay is crucial for an accurate derivation of the OAE phase at the eardrum. While measurements of the OAE phase slope in human ears tend to be noisy, there is still an advantage of applying the proposed corrections, particularly near the half-wave resonance frequencies (FIG. 6E) so that the sensitivity of the OAE phase to the residual ear-canal acoustics is reduced.

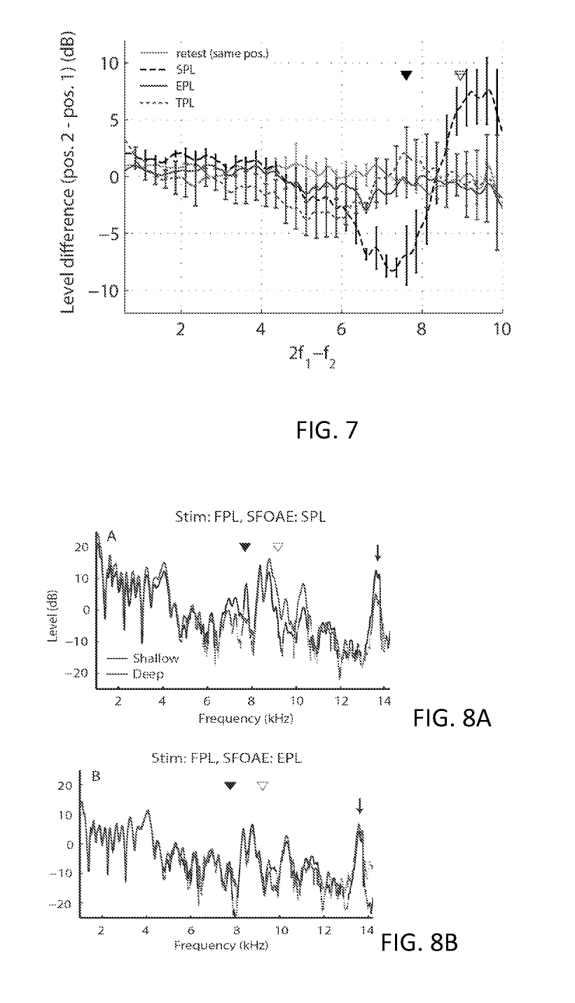

To assure the observations made in FIG. 6E also hold in a larger sample size, additional DPOAE data were collected in four additional normally hearing human subjects. The average change (n=5) in the DPOAE level due to change in the OAE probe position (deep vs. shallow) is shown in FIG. 7 for different OAE metrics and compared to the change in the DPOAE level measured for the same probe position (grey—the estimate of test retest repeatability that is not related to changes in the acoustic load). The data points with signal-to-noise ratio less than 6 dB were excluded from the calculations (16 to 19% of data points depending on the curve). For clarity the curves were gently smoothened and error bars (±1 standard error of the mean) only every 0.25 kHz are displayed.

Both metrics proposed in this invention (P_{EPL}—solid red and P_{TPL}—dotted red) diminish the sensitivity of the DPOAE to a change in the acoustic load to nearly the measurement test-retest level. In theory, P_{TPL} is completely independent of the acoustic load (both related to the ear canal and probe source), while OAE_{EPL} depends on the characteristic impedance of the ear canal (i.e., it's cross sectional area). Thus, P_{TPL} may be a more appropriate metric when comparing OAEs across multiple subjects (i.e., with different diameters of ear canals). In our sample, P_{TPL} did show decreased sensitivity to the probe insertion depth (FIG. 7, dotted red) as compared to the conventional measures (dashed black), but its performance tended to be worse in the mid frequency range as compared to P_{EPL} (solid red). We also observed more variability in the P_{TPL} performance in correcting for the ear canal acoustics across the subjects (see the size of the error bars), while both methods performed similarly in the cavity test (FIG. 6B). This slightly lower performance indicates that this metric may be more susceptible to inaccuracies in determining the in situ ear canal and probe reflectance.

Overall, these results demonstrate that compensating for the effects of ear-canal acoustics on both the evoking stimuli and the resulting emissions allows OAE measurements to be made reproducibly across test sessions, independent of probe placement in the ear canal, over frequencies spanning most of the range of human hearing.

### Example 3—Application to Other OAE Types

Although we focus here on the application of emitted pressure to DPOAE measurements, the conversion to emitted pressure using the methods described herein can be applied to any type of OAE whenever the ear-canal and probe-source reflectances are known.

FIGS. 8A and 8B show the results of applying emitted pressure to stimulus-frequency OAEs (SFOAEs). The SFOAEs were evoked using FPL-calibrated tones at two probe locations in the ear canal (shallow=black vs. deep=red) both before (FIG. 8A) and after (FIG. 8B) conversion to emitted pressure level (EPL). Data segments with SNR<6 dB are shown using dotted lines. SFOAEs were measured using the interleaved suppression method at frequencies swept from 1-16 kHz at 1 oct/sec (Kalluri and Shera, “Measuring stimulus-frequency otoacoustic emissions using swept tones,” J. Acoust. Soc. Am., 134:356-368. (2013)). Probe and suppressor levels were 37 dB FPL and 57 dB FPL, respectively. Triangles mark the half-wave resonances and the arrow indicates the frequency of a strong spontaneous OAE.

When expressed in the conventional way (P_{SPL}) as shown in FIG. 8A, the measurements show a dependence on insertion depth similar to that seen with DPOAEs (i.e., level shifts of 2-3 dB at low frequencies and ˜10 dB near f_{λ/2}). In contrast, as shown in FIG. 8B, SFOAEs expressed using emitted pressure level (P_{EPL}) are nearly unaffected by probe position, even at frequencies above 10 kHz (see the arrow).

Similarly, the use of emitted pressure appears equally effective at removing the dependence on ear-canal acoustics from transient-evoked (TE) OAEs (same as in FIGS. 8A and 8B, data not shown). For simplicity, we employed FPL-shaped clicks for the measurements of TEOAEs (Scheperle et al., “Further assessment of forward pressure level for in situ calibration,” J. Acoust. Soc. Am., 130:3882-3892 (2011)). However, the calibration of transient stimuli needs to be carefully considered based on the duration of the stimulus. For example, when the duration of the transient is comparable to or less than the round-trip ear-canal delay, calibration procedures based on the steady-state response, such as FPL, are likely inappropriate. Instead, calibrations that equalize the initial outgoing stimulus pressure (the “emitted stimulus”) may be the better choice (e.g., Goodman et al., “High-frequency click-evoked otoacoustic emissions and behavioral thresholds in humans,” J. Acoust. Soc. Am., 125:1014-1032 (2009)).

These results demonstrate that the methods described herein to convert to emitted pressure can be applied to any type of OAE whenever the ear-canal and probe-source reflectances are known, e.g., not only OAEs evoked using two tones (as illustrated in Example 2 (and in FIGS. 6A-F and 7, but also for OAEs evoked using other stimuli (e.g., by a single tone).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for measuring otoacoustic emissions (OAEs) in a subject using an OAE probe, wherein the measurement is corrected for the subject's ear canal acoustics and for the OAE probe, the method comprising:
(a) inserting the OAE probe into the subject's ear canal;
(b) delivering a calibration stimulus into the ear canal with the OAE probe and detecting any calibration signal propagated from within the ear canal;
(c) using the detected calibration signal to calculate calibration measurements comprising ear canal reflectance, ear canal one-way delay, and OAE probe reflectance;
(d) delivering an excitation stimulus sufficient to evoke an OAE into the ear canal with the OAE probe;
(e) collecting any OAE response;
(f) converting the OAE response using the calculated calibration measurements from step (c) into an unbiased OAE response; and
(g) displaying the unbiased OAE response.

2. The method of claim 1, wherein the calibration signal is further used to calibrate the excitation stimulus used to evoke the OAE.

3. The method of claim 1, wherein the excitation stimulus is a wide-band chirp that covers the range of frequencies within the human audible range.

4. The method of claim 1, wherein detecting any calibration signal emitted from within the ear canal comprises detecting a pressure from within the ear canal.

5. The method of claim 1, wherein converting the OAE response comprises correcting OAE amplitude and phase.

6. The method of claim 5, wherein correcting OAE amplitude and phase comprises calculating emitted pressure (P_{EPL}) or Thévenin-equivalent source pressure (P_{TPL}) using the calibration measurements.

7. The method of claim 6, wherein the OAE response measured at the OAE probe (P_{SPL}) is converted to emitted pressure (P_{EPL}) using the equation:
$${P}_{EPL} = {P}_{SPL} \frac{( 1 - {R}_{EC} {R}_{s} )}{t ( 1 + {R}_{s} )}$$
where R_{EC} is the ear-canal reflectance, R_{S} is the OAE probe reflectance, and t is equal to e^{-2πfτ}, with τ corresponding to one-way ear canal delay.

8. The method of claim 6, wherein the OAE response measured at a microphone in the OAE probe (P_{SPL}) is converted to Thévenin-equivalent source pressure (P_{TPL}) using the equation:
$${P}_{TPL} = {P}_{SPL} \frac{2 t ( 1 - {R}_{EC} {R}_{s} )}{( 1 + {R}_{s} ) ( {t}^{2} - {R}_{EC} )}$$
where R_{EC} is the ear-canal reflectance, R_{S} is the OAE probe reflectance, and t is equal to e^{-2πfτ}, with τ corresponding to one-way ear canal delay.

9. The method of claim 1, further comprising using the displayed unbiased OAE response to determine the health of the inner ear of the subject.

10. A method for calculating complex otoacoustic emission (OAE) emitted sound pressure (P_{EPL}) at the eardrum, equivalent to a complex OAE pressure measured in an anechoic ear canal, the method compromising:
(a) measuring the complex OAE sound pressure (P_{SPL}) with an OAE probe microphone coupled to the ear canal;
(b) measuring the ear canal reflectance (R_{EC}), OAE probe reflectance (R_{S}), and one-way ear canal delay (τ) using the same probe position used in the P_{SPL} measurements; and
(c) at any frequency f calculating the P_{EPL} according to:
$${P}_{EPL} = {P}_{SPL} \frac{( 1 - {R}_{EC} {R}_{s} )}{t ( 1 + {R}_{s} )} , \\ where t = {e}^{- i 2 π f τ} .$$

11. A method for calculating a load-independent Thévenin-equivalent complex OAE source pressure at the eardrum (P_{TLP}), the method compromising:
(a) measuring the complex OAE sound pressure (P_{SPL}) with an OAE probe microphone coupled to the ear canal;
(b) measuring the ear canal reflectance (R_{EC}), OAE probe reflectance (R_{S}), and one-way ear canal delay (τ) using the same probe position used in the P_{SPL} measurements; and
(τ) at any frequency f calculating the P_{TLP} according to:
$${P}_{TPL} = {P}_{SPL} \frac{2 t ( 1 - {R}_{EC} {R}_{s} )}{( 1 + {R}_{s} ) ( {t}^{2} - {R}_{EC} )} , \\ where t = {e}^{- i 2 π f τ} .$$

12. The method of claim 1, further comprising a preliminary step of calibrating the OAE probe itself in a set of dummy loads before inserting the OAE probe into the subject's ear.

13. The method of claim 1, wherein the subject is a human.

14. The method of claim 13, wherein the human is an infant or an adult.
